(19) United States
(12) Patent Application Publication
(10) Pub. No.: US 2010/0081999 A1
(43) Pub. Date: Apr. 1, 2010

(54) SYRINGE, IN PARTICULAR FOR VETERINARY-MEDICINE APPLICATIONS

(75) Inventors: Ralf Rueckert, Wurmlingen (DE); Roland Keuffel, Neuhausen ob Eck (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: HENKE-SASS, WOLF GMBH, Tuttlingen (DE)

(21) Appl. No.: 12/567,393

(22) Filed: Sep. 25, 2009

(30) Foreign Application Priority Data
Sep. 26, 2008 (DE) 102008049082.2

(51) Int. Cl. A61M 5/32

(52) U.S. Cl. 604/198

(57) ABSTRACT

A syringe for veterinary-medicine applications has a main body with a syringe cylinder whose discharge-side end is connected to a cannula and in which a piston connected to a piston rod is situated so it is displaceable. The syringe includes a protective module including protective cap and a securing element. The protective cap is mounted so it is displaceable on the main body, which receives the cannula in its first terminal position so that the front end thereof does not protrude from the protective cap. The securing element can be moved from a first securing position into a second securing position and then into a third securing position. The securing element releases a locking of the piston rod relative to displacements, so that the piston can be moved in the syringe cylinder in the direction toward the discharge-side end using the piston rod.

### PRIORITY APPLICATION

The current application claims the benefit of priority to German Patent Application No. DE 10 2008 049 082.2 filed on Sep. 26, 2008. Said application is incorporated in its entirety by reference herein.

### FIELD OF THE INVENTION

The present invention relates to a syringe, in particular for veterinary-medicine applications, having a main body which has a syringe cylinder, whose discharge-side end is connected to a cannula and in which a piston connected to a piston rod is situated so it is displaceable.

### BACKGROUND OF THE INVENTION

Such syringes are used, for example, in the field of veterinary medicine for injecting medications. For this purpose it is important to ensure that the medication is not unintentionally injected in humans.

Proceeding therefrom, it is the object of the invention to refine the syringe of the type cited at the beginning so that unintentional injection of a fluid present in the syringe cylinder can be reliably avoided.

### SUMMARY OF THE INVENTION

The object is achieved in a syringe of the type cited at the beginning in that the syringe has a protective module, which has a protective cap mounted so it is displaceable on the main body, which receives the cannula in its first terminal position so that the front end of the cannula does not protrude from the protective cap, and a securing element, which can be moved from a first securing position into a second securing position and then into a third securing position, the securing element, in its first securing position, locking the protective cap in its first terminal position, the protective cap being displaceable toward the main body into its second terminal position, in which the front end of the cannula protrudes out of the protective cap, if the securing element is in the second securing position, and the securing element, in its third securing position, into which it can only be moved from the second securing position if the protective cap is in its second terminal position, releases a locking of the piston rod relative to displacements, which is caused by the protective module in the first and second securing positions, so that using the piston rod, the piston can be moved in the syringe cylinder in the direction toward the discharge-side end.

It is thus ensured in the syringe according to the invention, when the protective cap is in its first terminal position and the securing element is in its first securing position, that the cannula cannot be unintentionally stuck in, because its front end lies completely inside the protective cap.

The protective module is preferably implemented so that in its basic position, the protective cap is in its first terminal position and the securing element is in the first securing position. In particular, the first terminal position of the protective cap and the first securing position of the securing element may be bias tensioned by elastic elements so that an application of force is necessary to bring the cannula out of the first terminal position and the securing element out of the first securing position.

Even if the securing element is in its second securing position and the cannula is unintentionally stuck in, an application of the fluid located in the syringe cylinder is still prevented in the syringe according to the invention, because the piston rod is locked both in the first and also in the second securing position of the securing element against displacements because of the protective module. The piston rod can only be moved when the securing element is in its third securing position. Therefore, a three-stage security mechanism is provided, which reliably prevents unintentional application of a fluid from the syringe cylinder.

Because the syringe according to the invention is implemented so that the securing element may be moved from the second securing position into the third securing position only if the protective cap is in its second terminal position, the necessary piercing depth of the cannula can thus be permanently predefined. An application of the fluid from the syringe cylinder is only possible when this piercing depth has been reached. It can thus be easily ensured that the application of the fluid, which can particularly be a medication, also has the desired effect.

The securing element can be implemented as a lever mounted so it is pivotable in the main body. It is thus easily possible to bring a securing element into three different securing positions.

In particular, the securing element can have a locking section, which causes the locking of the piston rod in the first and second securing positions.

Thus, for example, the locking section may press two balls mounted in the main body into a groove in the piston rod in the first and second securing positions. The groove can particularly be a radial peripheral groove. Secure locking and/or blocking of the piston rod against displacements is possible using such a locking section.

The locking section can be implemented as essentially U-shaped having two free legs, the spacing of the two legs changing toward the open end of the U-shaped locking section. The spacing can increase or decrease and is selected so that the balls are reliably pressed into the groove in the first and second securing positions and the spacing is so great in the third securing position that the balls may be pressed out of the groove, so that the locking is released.

The protective cap can have a front section and a main section in the syringe according to the invention, the front section being removably connected to the main section and having a cannula tool, using which the cannula can be removed from the syringe cylinder.

The cannula tool is, for example, a special implementation of the front opening of the front section. In this case, the front opening may be pushed over the cannula up to the socket section of the cannula, a form fit then existing between the opening and the socket section, so that pivoting is possible using the front section. Alternatively, it is possible to implement a formfitting tool for the socket section of the cannula on the outer wall of the front section.

The protective cap in particular has a front opening through which the tip of the cannula runs during the movement of the protective cap from its first into its second terminal position.

This syringe can also have a compression spring between the piston rod and the main body, the piston rod being brought into its starting position using the compression spring, in which it is locked against displacements in the first and second securing positions of the securing element.

Furthermore, the syringe can have an attachment for filling. The attachment can in particular be implemented having a check valve, so that the backward movement of the piston after the discharge results in filling of the syringe cylinder via the filling attachment. The filling attachment can have a bottle holder for receiving a medication container or also a hose attachment.

It is understood that the above-mentioned features and the features to be explained hereafter are usable not only in the disclosed combinations, but rather also in other combinations or alone without leaving the scope of the present invention.

### BRIEF DESCRIPTION OF THE DRAWINGS

The invention is explained in greater detail for exemplary purposes hereafter on the basis of the appended drawings, which also disclose features essential to the invention. In the figures:

FIG. 1 shows a sectional view of a first embodiment of the syringe according to the invention.

FIG. 2 shows a side view of a second embodiment of the syringe according to the invention having a partial sectional illustration of the pin.

FIG. 3 shows an illustration of the syringe from FIG. 2, the security lever being in its second pivot position.

FIG. 4 shows an illustration of the syringe from FIG. 2, the syringe being stuck into an injection point.

FIG. 5 shows a view of the syringe from FIG. 2, the security lever being in its third pivot position.

FIG. 6 shows an illustration of the syringe from FIG. 2, the security lever being in its third securing position and the piston rod being moved toward the main body 2 to discharge the medium from the syringe cylinder.

FIGS. 7a-7c show perspective partial views to explain the locking of the piston rod.

FIGS. 8a-8c show sectional views from the rear to explain the locking of the piston rod.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF EMBODIMENTS OF THE INVENTION

In the embodiment shown in FIG. 1, the syringe 1 according to the invention comprises a main body 2, in which a syringe cylinder 3 is situated. The syringe cylinder 3 is connected at the discharge side via a channel 4 and a check valve 5 to a cannula 6.

The syringe 1 additionally comprises a protective cap 7 having a front opening 8, the protective cap 7 being mounted on the main body 2 so it is displaceable in the axial direction. The protective cap 7 is in its first terminal position in FIG. 1, in which the tip 9 of the cannula 6 does not protrude forward out of the protective cap 7.

A piston 10, which is displaceable in the longitudinal direction, is situated in the syringe cylinder 3, the piston being connected to a piston rod 11 guided in the main body 2. The piston rod 11 protrudes out of the main body 2 to the rear and is connected to a handle element 12. The starting position of the piston 10 and piston rod 11 is shown in FIG. 1.

A security lever 14, which is pivotable around an axis 13, is situated on the top side of the main body 2, which can be put in to three different securing/pivot positions, as described in detail hereafter.

In the illustration of FIG. 1, the security lever 14 is in its first pivot position, in which its front end 15 presses against a stop 16 of the protective cap 7, so that the protective cap 7 cannot be displaced toward the main body 2. In order to keep the security lever 14 in its first pivot position, a coiled spring 18 is situated between a rear end 17 of the security lever 14 and the main body 2, which holds the security lever 14 in the first pivot position.

In this state, unintentionally sticking in the cannula 6 is reliably prevented because of the protective cap 7, which is in the first terminal position.

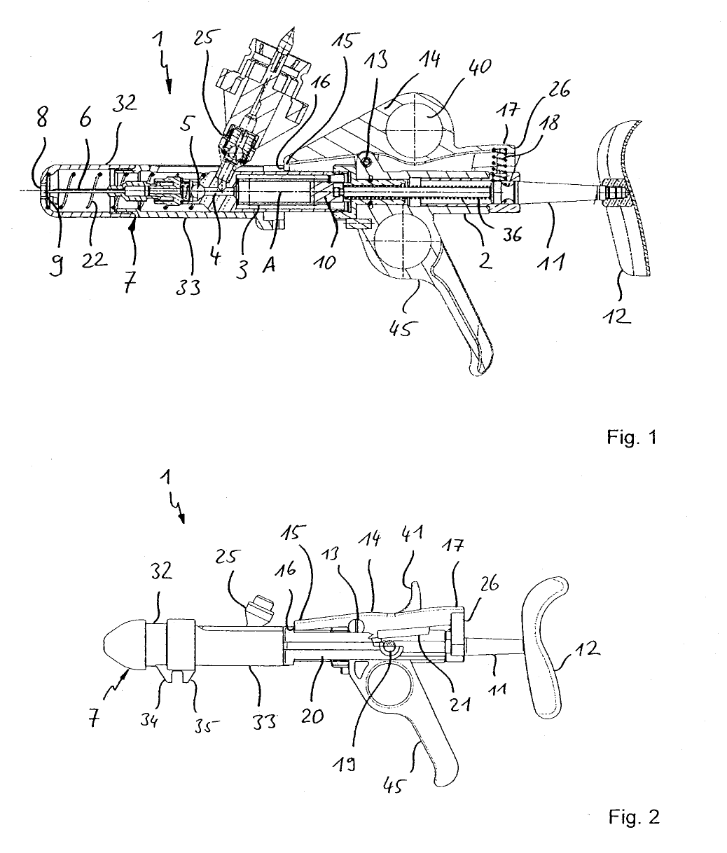

This is also true for the embodiment of the syringe 1 according to the invention shown in FIG. 2. The embodiment of FIG. 2 differs from that of FIG. 1 only through the implementation of the security lever 14 and the protective cap 7. In the embodiment of FIG. 1, the security lever 14 has a through hole 40 for the index finger of the operator. The security lever 14 of FIG. 2 has a finger projection 41, which protrudes upward, instead of the through hole 40, which the index finger can wrap around from the rear, for example, in order to bring the security lever 14 out of its first pivot position. Otherwise, the security levers 14 of FIGS. 1 and 2 are essentially identical and have the same functionality.

The protective caps 7 of FIGS. 1 and 2 are also essentially identical and have the same functionality. The protective cap 7 of FIG. 2 also has projections 34, 35 in comparison to the protective cap 7 of FIG. 1, whose function will be described later.

The syringe 1 also has a finger handle 45 on its bottom side.

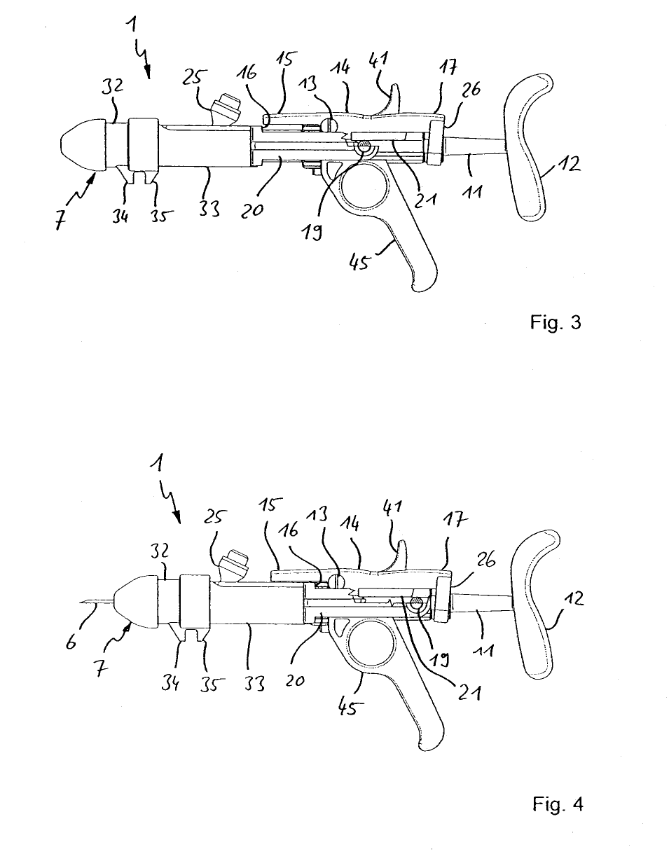

Upon usage of the syringe 1, an operator must first move the security lever 14 from its first pivot position (FIG. 1, FIG. 2) into its second pivot position, which is shown in FIG. 3 (the embodiment of the syringe from FIG. 2 is shown in FIGS. 3 through 6). For this purpose, the operator must merely press using his index finger in the rear area of the security lever 14 (i.e., between the axis 13 and the rear end 17) on the finger projection 41 of the security lever 14, so that the front end 15 and the stop 16 are disengaged, as shown in FIG. 3. The security lever 14, which is also referred to as a securing lever 14 hereafter, it thus moved by a pivot movement from the first securing position into the second pivot position. Because this pivot movement is performed against the spring force of the coiled spring 18, the operator must actively keep the security lever 14 in the second pivot position.

As shown in FIG. 3, the second pivot position is fixed by a pin 19 running perpendicularly to the axial axis A of the syringe 1, which is connected via an intermediate element 20 to the protective cap 7, and against which a lower stop rail 21 of the security lever 14 presses. Because the stop rail 21 is behind the intermediate element 20 in the illustrations of FIGS. 2-6 and would therefore be concealed thereby, the pin 19 is shown in a partial sectional illustration in FIGS. 2-6. A pin retained by an intermediate element 20 is also provided on the side of the syringe 1 not visible in FIG. 3, which works together with a further stop rail of the security lever 14.

If the operator now moves the security lever 14 into the second securing position shown in FIG. 3 and holds it, he can place the syringe 1 on the injection point (e.g., on an animal, not shown here) and move it forward. In this case, the protective cap 7 will remain at the position defined by the injection point, while the main body 2 is moved relative to the protective cap 7 and thus also the cannula 6 is moved relative to the protective cap 7 and the cannula 6 is thus pierced into the injection point. The protective cap 7 is thus pushed back over the cannula 6. A predetermined application of force is necessary for this purpose because of a coiled spring 22 situated between the main body 2 and the protective cap 7.

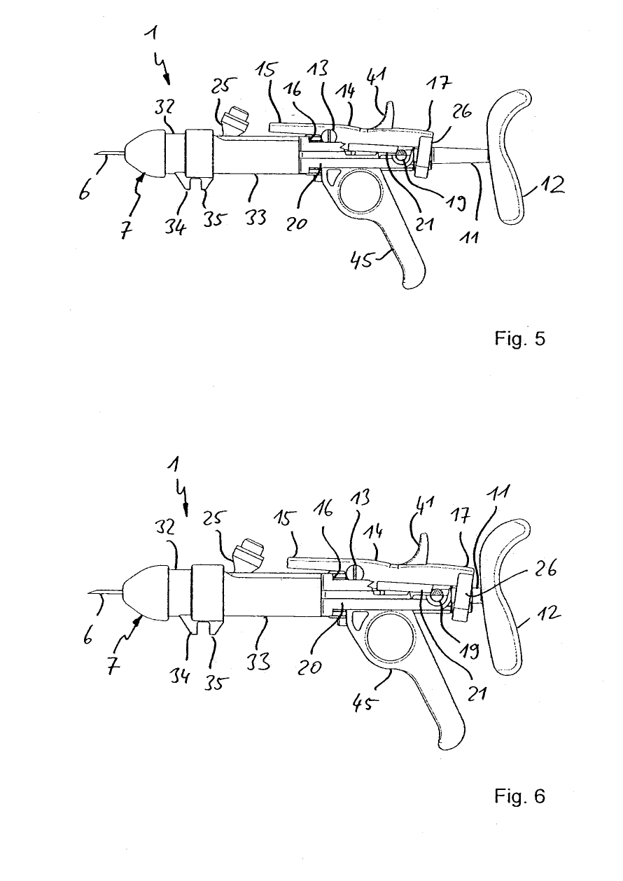

During this relative movement between main body 2 and protective cap 7, the front end 15 of the security lever 14 slides on the top side of the protective cap 7 and the stop rail 21 slides on the pin 19, so that the security lever 14 remains in its second securing position and cannot be brought into the first or the third securing position. If the pin 19 is displaced enough relative to the stop rail 21 that it lies behind the rear end of the stop rail 21 and thus no longer predetermines the second securing position (FIG. 4), the operator can move the security lever 14 from its second securing position by rotation around the axis 13 into its third securing position shown in FIG. 5.

The third securing position 11 differs from the first and second securing positions in that the piston rod 11 is no longer locked, but rather can be moved in the axial direction. The operator can now move the piston rod 11 in the direction toward the main body 2 (FIG. 6), whereby the piston 10 is moved in the syringe cylinder 3 toward the discharge-side end. The medication located in the syringe cylinder 3 is thus introduced via the cannula 6 into the injection point.

The medication can thus first be applied using the syringe 1 according to the invention when the protective cap 7 is in its second terminal position and the security lever 14 is in its third securing position. Unintentional application of the medication is thus prevented. Furthermore, the piercing depth is predetermined by the second terminal position of the protective cap 7. The medication can first be applied upon reaching the predetermined piercing depth, whereby the introduction of the medication at the predetermined piercing depth can be ensured.

When the syringe 1 is withdrawn from the injection point after introduction of the medication, the protective cap 7 slides back into its first terminal position because of the coiled spring 22, the piston rod 11 and thus the piston 10 also being brought into its starting position simultaneously because of a piston spring 36. Furthermore, the security lever 14 is pivoted into its first securing position because of the coiled spring 18. The syringe 1 is thus located in the state shown in FIGS. 1 and 2 again, in which unintentional sticking using the cannula is not possible.

Because of the reverse movement of the piston rod 11, the next medication dose to be applied is sucked into the syringe cylinder 3 in a known way via a feed 25 having check element, which opens into the channel 4.

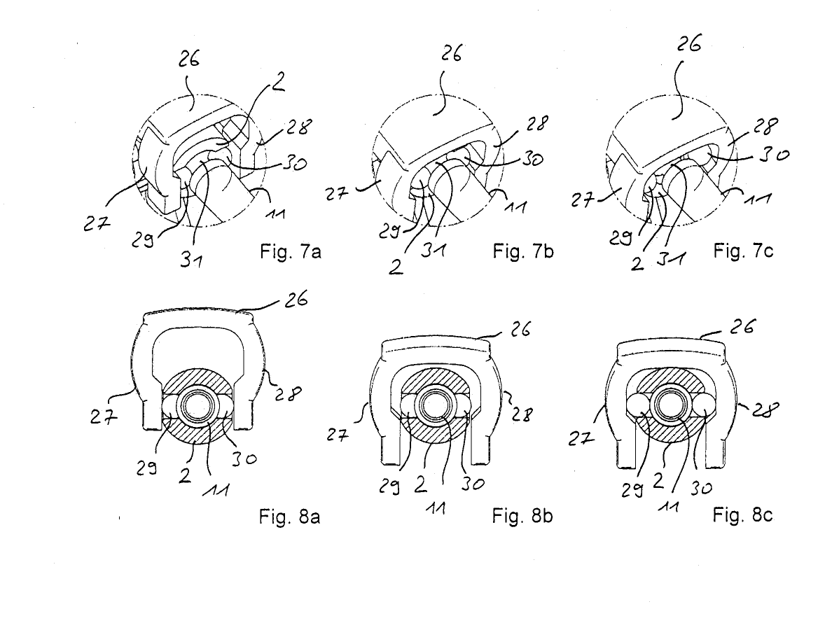

The locking of the piston rod 11 in the first and second securing positions and its release in the third securing position is described in connection with FIGS. 7a-7c and 8a-8c.

A downwardly open, U-shaped locking element 26 is implemented on the rear end of the securing lever 14, whose two free legs 27, 28 have a greater spacing from one another in the upper area than in the lower area. In the second securing position shown in FIGS. 7a and 8a, the two legs 27 and 28 press two balls 29, 30, which are situated in a ball seat implemented in a main body 2, into a radial peripheral groove 31 in the piston rod 11.

This is also true for the first securing position (not shown), in which the front part of the two legs 27, 28 also presses the balls 29, 30 into the groove 31. An axial movement of the piston rod 11 is thus blocked. Unintentional discharge of the medication from the syringe cylinder 3 is thus effectively prevented.

When the security lever 14 is in its third securing position, the balls 29 and 30 are no longer in the narrower area between the two free legs 27 and 28, but rather in the wider area. It is therefore now possible by sliding the piston rod 11 to press the two balls 29 and 30 outward in the radial direction, as shown in FIGS. 7c and 8c, so that the piston rod 10 is movable in the axial direction. The locking is thus canceled.

If the security lever 14 and thus the locking element 26 are brought out of the third securing position back into the second and the first securing positions, the two balls 29 and 30 are pressed back into the peripheral groove 31 in the piston rod 10 because of the free legs 27 and 28, whose spacing decreases toward the open end (of course, this is only possible if the piston rod 11 is back in its starting position), whereby the piston rod 10 is locked against movements in the axial direction again.

The protective cap 7 described here has a front section 32, which is screwed onto a rear cap section 33. After the front section 32 is unscrewed, the cannula 6, which is in turn removably connected to the syringe cylinder 3 (e.g., screwed on), can be replaced.

In the embodiment of FIGS. 2-6, the front section has the two projections 34, 35, which are selected in their shape and dimensions so that in the unscrewed state of the front section 32 having the projections 34 and 35, the cannula 6 can be unscrewed. A direct contact of the cannula with the skin of the user can thus be avoided. Of course, the projections 34, 35 may also be used for screwing on a new cannula.

In the embodiment of the syringe 1 shown in FIG. 1, the front opening 8 is implemented so that the unscrewed front section 32 can be turned around and pushed over the cannula 6 and over the cannula base with the front opening 8 first, a form fit existing between the opening 8 and the cannula base. The front section 32 can thus again be used as a tool for loosening and/or fastening the cannula 6.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents.

1. A syringe for veterinary-medicine applications, comprising:
a main body comprising a syringe cylinder with a discharge-side end connected to a cannula, and a piston displaceable in the syringe cylinder, the piston connected to a piston rod;
a protective module having a protective cap mounted so it is displaceable on the main body, the protective cap receiving the cannula in a first terminal position so that a front end thereof does not protrude from the protective cap;
the protective module also including a securing element that is movable from a first securing position into a second securing position and then into a third securing position;
the securing element in its first securing position locking the protective cap in the first terminal position;
the protective cap being displaceable toward the main body into a second terminal position in which the front end of the cannula protrudes from the protective cap if the securing element is in the second securing position; and
the securing element, in a third securing position into which it is only movable from the second securing position when the protective cap is in its second terminal position, releasing a locking of the piston rod relative to displacement, which is caused in the first and second securing positions by the protective module, so that the piston can be moved in the syringe cylinder in the direction toward the discharge-side end using the piston rod.

2. The syringe according to claim 1, wherein the securing element is implemented as a lever mounted so it is pivotable on the main body.

3. The syringe according to claim 1, wherein the protective cap is spring-loaded in its first terminal position.

4. The syringe according to claim 2, wherein the protective cap is spring-loaded in its first terminal position.

5. The syringe according to claim 1, wherein the securing element is spring-loaded in the first securing position.

6. The syringe according to claim 2, wherein the securing element is spring-loaded in the first securing position.

7. The syringe according to claim 3, wherein the securing element is spring-loaded in the first securing position.

8. The syringe according to claim 1, wherein the securing element has a locking section, which causes the locking of the piston rod in the first and second securing positions.

9. The syringe according to claim 8, wherein the locking section presses two balls, which are mounted in the main body, into a groove in the piston rod in the first and second securing positions.

10. The syringe according to claim 8, wherein the locking section is implemented as essentially U-shaped having two free legs, the spacing of the two legs changing toward the open end of the U-shaped locking section.

11. The syringe according to claim 9, wherein the locking section is implemented as essentially U-shaped having two free legs, the spacing of the two legs changing toward the open end of the U-shaped locking section.

12. The syringe according to claim 1, wherein the protective cap has a front section and a main section, the front section being removably connected to the main section and having a cannula tool, using which the cannula can be removed from the syringe cylinder.

13. A syringe having
a main body, which has a syringe cylinder, whose discharge-side end is connected to a cannula and in which a piston, which is connected to a piston rod, is situated so it is displaceable,
and a protective module, which has
a protective cap, mounted so it is displaceable on the main body between a first terminal position and a second terminal position, receives the cannula in its first terminal position so that the front end thereof does not protrude from the protective cap, and
a securing element, which can be moved from a first securing position into a second securing position and then into a third securing position,
said protective module locks the piston rod relative to displacements, if said securing element is in the first or second securing position,
the securing element, in its first securing position, locking the protective cap in its first terminal position,
the protective cap being displaceable from the first terminal position into its second terminal position, in which the front end of the cannula protrudes from the protective cap, if the securing element is in the second securing position,
and the securing element, in its third securing position, into which it is only movable from the second securing position when the protective cap is in its second terminal position, releasing said locking of the piston rod relative to displacements, which is caused in the first and second securing positions by the protective module, so that the piston can be moved in the syringe cylinder in the direction toward the discharge-side end using the piston rod.

14. The syringe according to claim 13, being adapted for veterinary-medicine application
